(19) United States
(12) Patent Application Publication
(10) Pub. No.: US 2017/0233719 A1
(43) Pub. Date: Aug. 17, 2017

(54) METHODS FOR SEPARATING NUCLEIC ACIDS WITH GRAPHENE COATED MAGNETIC BEADS

(71) Applicant: Life Magnetics, Inc., Ann Arbor, MI (US)

(72) Inventors: Kevin Hagedorn, Ann Arbor, MI (US); Saravana Murthy, Gaitersburg, MD (US)

(21) Appl. No.: 15/434,470

(22) Filed: Feb. 16, 2017

Related U.S. Application Data
(60) Provisional application No. 62/295,985, filed on Feb. 16, 2016.

(51) Int. Cl. C12N 15/10

(52) U.S. Cl. CPC C12N 15/1013

(57) ABSTRACT

Disclosed herein are methods for separating and identifying nucleic acids by utilizing carbon coated magnetic beads. The method teaches that multivalent cations promote binding of single stranded nucleic acids to the beads and that the single stranded nucleic acids can be released with the addition of chelating agents that bind the multivalent cations such as EDTA. The method further teaches that fragile single stranded nucleic acids, such as RNA, can be stored on the surface of the beads. Lastly, the method also teaches that by iteratively adding complimentary DNA oligos, single stranded nucleic acids can be quantified or individually isolated using the carbon coated magnetic beads.

### BACKGROUND

This disclosure claims priority to U.S. Provisional Application Ser. No. 62/295,985, filed Feb. 16, 2016 entitled Methods for Separating Nucleic Acids with Graphene Coated Magnetic Beads, the specification of which is incorporated by reference herein in its entirety.

This disclosure relates to nucleic acid purification and isolation, magnetic bead biological separations, and carbon and its allotropes

The need for effective techniques for isolating single stranded nucleic acid components from associated biological materials persists in a variety of research and development endeavors. Heretofore, nucleic acid separation techniques can include various chelating agents, solid support columns using fibrous or silica matrices to bind nucleic acids and/or magnetic beads to which nucleic acid can bind. The isolation methods presently in use do not provide effective yield in all instances. Thus it would be desirable to provide a method and material that can provide more effective and efficient nucleic acid isolation.

### SUMMARY

A method for separating single stranded nucleic acids from a sample that includes the steps of providing a mixture containing nucleic acids: and providing carbon coated material and at least one of the following: Group 2 cations, transition metal cations, Y⁺ cations such as guanidinium thiocyanate and creating a complexes of the carbon coated material and nucleic acids. The method disclosed herein can also include the step of removing the carbon coated material complexed with nucleic acids from the resulting mixture. In certain embodiments, the carbon-coated material employed can be in the form of magnetic beads, silica gel or various polymeric materials.

In certain embodiments, carbon coated magnetic beads may be employed that can have a magnetic core coated surrounded by a suitable carbon material. In other embodiments, the carbon coated material may have a non-magnetic core such as silica coated with a suitable carbon material.

### BRIEF DESCRIPTION OF THE DRAWINGS

The various features, advantages and other uses of the present apparatus will become more apparent by referring to the following detailed drawings.

FIG. 1 is a schematic drawing for how single stranded nucleic acids are isolated.

FIG. 2 is a flow chart of the process for isolating or purifying nucleic acids.

FIG. 3 is a schematic drawing for how a single stranded nucleic acid in a sample may be isolated and may also be quantified.

FIG. 4 is a flow chart for the process of how a single stranded nucleic acid in a sample may be isolated and may also be quantified.

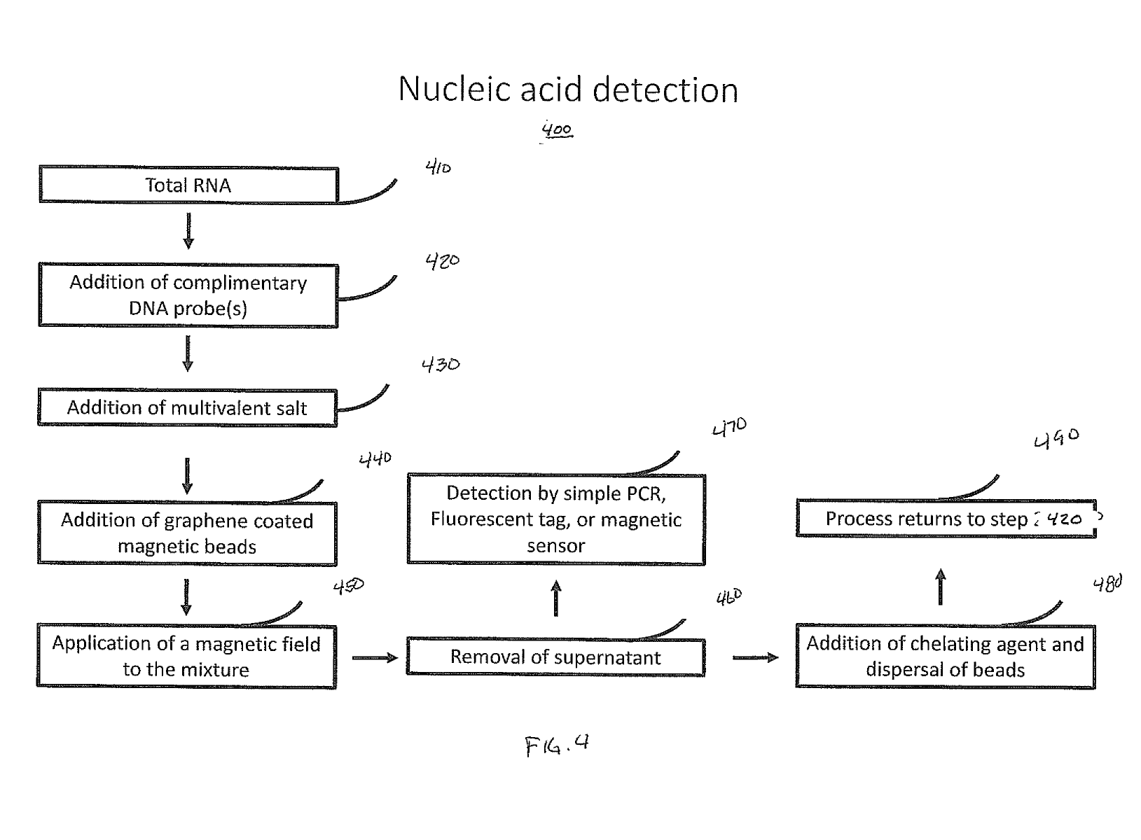

FIG. 5 is a transmission electron micrographs of a carbon coated magnetic beads which may be used in this process.

### DETAILED DESCRIPTION

The present disclosure is directed to a method for separating single stranded nucleic acids from an associated sample and can be employed to isolate nucleic acid material possessing specific sequencing. As broadly disclosed, the method involves a process in which a mixture containing nucleic acids is provided with or brought into contact with carbon-coated materials in the presence of cations selected from the group consisting of Group 2 elements, transition metals, salt, guanidinium thiocyanate, and mixtures thereof to create a carbon-nucleic acid complex.

The carbon coated material can be composed of a suitable substrate having an outer surface with a suitable carbon material covering at least a portion of the outer surface of the substrate. The resulting carbon-nucleic acid complex may be separated from the remaining materials by various separation techniques. The techniques employed can be chosen based on factors such as the nature of the of the substrate material employed.

It is contemplated that the process and matrix disclosed herein can be employed to isolate single-stranded nucleic acid with specific desired sequencing in certain embodiments. In such embodiments, the mixture of nucleic acids to be complexed can be the result of a suitable single stranded nucleic acid separation technique. The processes as disclosed herein can begin with a mixture of single stranded nucleic acids. These materials can be the result of suitable processes in which single stranded nucleic acids are isolated from a mixture of associated biological material. In such processes, a nucleic acid with sequence complimentary to the target, called the probe, is added to the mixture and the target and probe react to create a double stranded nucleic acid. Upon addition of Group 2 or transition metal cations, salt, or guanidinium thiocyanate, which can be referred to as a binding buffer, excess probes and unreacted nucleic acids create a complex of the carbon coated material and nucleic acids. Double stranded nucleic acid material that consist of one strand of target material and one strand of probe material remain in solution and are amenable to physical separation from other nucleic acid material. as used herein the term “nucleic acid probe” refers to a chain of nucleic acids selected so that it contains bases that are complementary with corresponding positions in at least one section of the length of the nucleic acid chain under study. The complimentary nucleic acid probe may be longer than the target and have additional bases with an arbitrary sequence.

Also disclosed is a composition that comprises a carbon coated substrate and single-stranded nucleic acid complexed therewith.

The carbon coated material as disclosed herein is one that will complex with single stranded nucleic acid material present in a sample. The single stranded nucleic acid material can include various naturally derived and/or synthetically prepared nucleic acids such as RNA, including any added complimentary nucleic acid probes if the target for these probes is not present in the sample under study. Particles with the single-stranded nucleic acids bound to the magnetic beads may be removed by application of a magnetic field, centrifugation, precipitation and the like.

The substrate material can be a configured in a suitable geometry such as particles or beads. The substrate material can be composed in whole or in part of magnetic materials, silica, polymeric materials and the like. It is contemplated that the method can encompass the use of suitable nucleic acid probes possessing target specific sequence will be those that remain in solution and will not form complexes with the carbon coated magnetic beads present and will form duplexes with the added complimentary nucleic acid probe, representing the isolated nucleic acids with the desired sequence. Additionally, the process may include the step of quantifying the single-stranded nucleic acid with the target sequence by performing suitable analytical processes such as PCR on duplex material that remains in solution using the complimentary DNA probe as a primer. Where desired or required, the complimentary nucleic acid probe may also be modified with a fluorescent tag. The fluorescent tag may be a may be a nanoparticle which is optically active, such as a surface-enhanced fluorescent particle.

In certain embodiments, the carbon coated material will include a substrate that is configured as a particulate material such as beads, or other spheroid bodies. When the substrate is configured as beads or other particulate geometries that are nanoparticulate in size; having a diameter between 1 and 100,000 nm and between 20 nm and 100,000 nm in certain embodiments. It is also contemplated that the beads can have an average particle diameter between 20 and 100,000 nm and between 50 and 200 nm in certain embodiments.

The process disclosed herein can be implemented using beads having magnetic characteristics where desired or required. Such magnetic beads can be configured with a magnetic core material having an outer surface and carbon material covering at least a portion of the outer surface. The magnetic beads can be nanoparticulate in size. It is contemplated that the magnetic beads employed can have a diameter between 1 nm to 100,000 nm with an average size diameter between 20 nm and 100,000 nm. In certain embodiments, the magnetic beads can have a size between 1 and 10,000 nm with an average diameter between 50 nm and 200 nm.

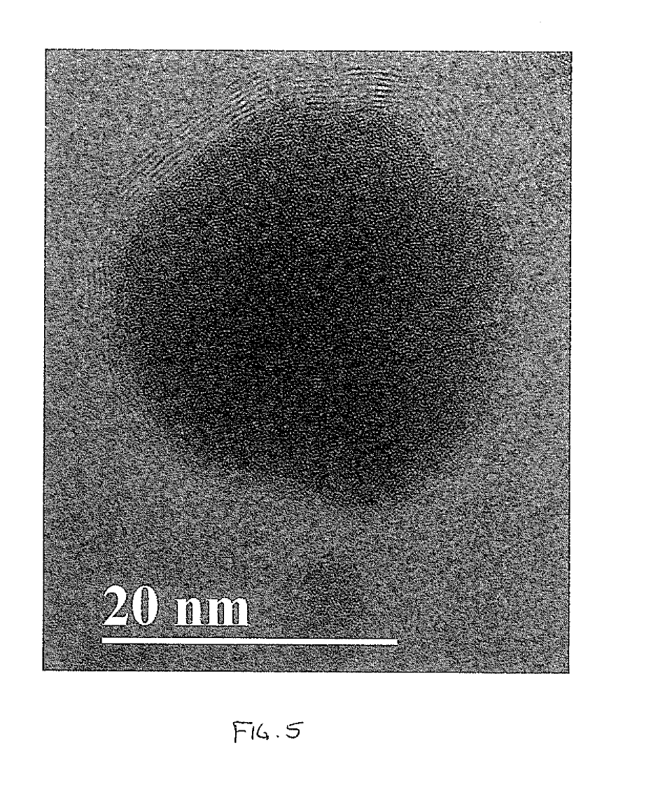

In certain embodiments, the carbon material can include one of more of the following: pyrolytic carbon, graphite, graphene. In certain embodiments, the suitable material is a material with a metal core that is surrounded by a carbon coating in which the carbon creates a generally seamless conformal coating. A suitable carbon coated material that can be used in the process as disclosed herein can be one such as the bead configuration presented in FIG. 5. Referring to FIG. 5, there is depicted a transmission electron micrograph in which a metal core composed of Fe₆₅Co₃₅ is surrounded by a carbon coating. This carbon coating has multiple layers, in which each layer is similar to graphene and there may be coupling between the layers. The presented carbon coated material structure is particularly well suited to the described process because the carbon creates a conformal coating without a seam around the metal core. Exposure of the metal core to water has undesirable effects due to oxidation of the metal. Oxidation of the metal may lead to undesirable properties such as binding DNA and reduction of magnetic moment. Suitable magnetic material that can be employed as the substrate is available commercially from Metastable Materials, Inc., doing business as Life Magnetics.

Carbon coated magnetic materials may be prepared by the laser manufacturing process disclosed in WO2015095398A1 which is disclosed herein by reference. It is contemplated that carbon coated materials can be prepared by positioning a laser is incident on target in a solvent such that the pulsed laser produces laser pulses having a pulse duration greater than 1 ps at a wavelength between 200 nm and 1500 nm at a pulse repetition rate of at least 10 Hz and a fluence greater than 10 J/cm². The laser beam may be scanned across the surface of the target; i.e., the desired core material (Silica, magnetic metal and the like). Non-limiting examples of satiable magnetic metal material includes metals and alloys containing iron, nickel, cobalt, or the like as well as mixtures and alloys of the aforementioned. The solvent employed can be an organic solvent which contains carbon and in certain embodiments will be composed of xylenes, toluene, benzene, or mixtures thereof. Where desired or required, the organic solvent may further include any hydrocarbon containing benzene rings. In certain embodiments, the magnetic beads employed will have a saturation magnetization between 10 and 80 A m²/kg (emu/g); a coercive force between 0.8 and 15.9 KA/m, and a sedimentation rate between 2% and 15% in 30 minutes.

In various embodiments disclosed herein, the substrate material such as the magnetic beads is coated with a suitable carbon based material. The carbon-based coating material affixed to the substrate may have a thickness between 1 and 50 angstroms and include effective amounts of carbon. In certain embodiments, the carbon material can be at least one of pyrolytic carbon, graphene or mixtures thereof. As the terms are employed herein, graphene is taken to be an allotrope of carbon consisting of a two-dimensional hexagonal lattice and graphite consists of stacked layers of graphene. Pyrolytic carbon is similar to graphite but with some covalent bonding between its graphene sheets. The carbons based coating material may also include certain amounts of graphitic oxide. As used herein, graphitic oxide is defined one or more of the foregoing carbon based materials exhibiting carboxylic acid groups and hydroxide groups formed at imperfections in the graphene sheet surface.

In the process as disclosed herein, the carbon coated material employed is present in a mixture with a suitable binding buffer. Suitable buffering binders include, but are not limited to a mixture of guanidinium thiocyanate, ethylenediaminetetraacetic acid (EDTA), tris(hydroxymethyl)aminomethane (Tris), dithiothreitol (DTT), and triton X. In certain embodiments, the binding buffer may be guanidinium thiocyanate, ethylenediaminetetraacetic acid (EDTA), tris(hydroxymethyl)aminomethane (Tris), dithiothreitol (DTT), and triton X and the solution may be adjusted to a pH of 6.5. Without being bound to any theory, it is believed that guanidinium thiocyanate may cause binding of the nucleic acids to the carbon at concentrations from 1M to 7M. EDTA and DTT may optionally be present to inactivate proteins and may be present in concentrations from 1 mmol to 100 mmol. Tris may optionally be present as a buffer to maintain the pH at 6.5. The pH may range from 4 to 9. The concentration of tris buffer may be from 20 to 200 mmol. Triton X is a detergent and may optionally be used to homogenize the solution. The concentration of Triton X is from 1 to 50 mmol. In certain embodiments, a suitable binding buffer may only require 1M to 7M guanidinium ions.

In the process as disclosed herein, the carbon coated material employed is in a mixture with Group 2 and/or transition metal cations. The Group 2 and/or transition metal cations employed may include an aqueous salt of one or more of the Group 2 and/or transition metal compounds. In certain embodiments, the cations present in the mixture include multivalent cations any of the Group 2 elements and/or transition metal compounds. Non-limiting examples of multivalent Group 2 cations include cations such as Be²⁺, Ca²⁺ and Mg²⁺ present alone or in combination. Non-limiting examples of transition metal cations include transition metals which form X²⁺ and/or X³⁺ cations when present in aqueous solution. These may include, but are not limited to Fe²⁺, Fe³⁺, Cu²⁺, Au³⁺, Sn⁴⁺, Sn³⁺, Cr²⁺, Ni³⁺, CO²⁺, Sn³⁺, Pt²⁺, Sr²⁺, or Ba²⁺. It is contemplated that one or more of the Group 2 cations can be present in the mixture alone or in combination with one or more of the transition metal cations.

In certain embodiments, it is contemplated that the mixture as disclosed herein is an aqueous composition that comprises between 1×10⁵ nanoparticulate beads per ml and 1×10²⁰ nanoparticulate beads per ml and between 0.1 mM and 10M of the cationic component. In the certain implementations of the process as disclosed herein, the mixture can contain 1×10⁸ beads per ml and 5 mmol of the cationic compound component. Where the cationic compound present includes a mixture of Group 2 compounds and transition metal compounds, it is contemplated that the cations will be present in a ratio of Group 2 compounds to transition metal compounds. In the certain specific embodiment, the cations present are Ca²⁺

Without being bound to any theory, it is believed that the mixture of carbon coated materials, such as carbon coated magnetic beads, and cations as disclosed herein, when brought into contact with nucleic acid constituents in the sample under analysis preferentially causes single stranded nucleic acids present in the sample to bind the surface of the beads creating a bio-complex. The resulting complex such as a magnetic bead and single stranded nucleic acid bio-complex is one which is amenable to removal by application of a magnetic field, precipitation, or other separation method. The resulting removed bio-complex is amenable to further operation and/or study.

Various methods can be employed in situations where it is considered desirable to release the single stranded nucleic acids from the surface of the carbon coated magnetic material present in the resulting bio-complex. In a first non-limiting example of a suitable method, the solid substrate may be separated from the liquid containing the Group 2 and/or transition metal cations or binding buffer and then water without these cations may be added, facilitating the release of the single stranded nucleic acids. In a second non-limiting example of a suitable method a chelating agent can be added to the isolated bio-complex in a subsequent operational step. Suitable chelating agents can include, but need not be limited to, various aminopolycarboxilic acids. In certain embodiments, chelators may include materials such as EDTA, BAPTA-AM, or EGTA. These chelating agents may be added as solids or dissolved in an aqueous solution.

The following terms are defined herein as follows:

A pellet refers to the magnetic beads compressed into a smaller volume. For example, if the beads are suspended in solution and a permanent magnet is placed in proximity to the vial, the beads are compressed from a dilute suspension to a pellet on the side of the vial.

The supernatant refers to the solution that remains from which the beads were extracted in the preceding example. The pellet is the extracted beads; the supernatant is the solution from which the beads were extracted.

The binding buffer refers to any combination of salts or cations which cause single stranded nucleic acids to bind to the carbon coated material to create a bio-complex.

A bio-complex refers to the single stranded nucleic acids bound to the carbon surface.

One non-limiting example of a method of isolating single stranded nucleic acids from biological samples using the method and material as disclosed herein employ graphene coated magnetic beads as disclosed and a controlled concentration of the multivalent salt or binding buffer. It has been unexpectedly discovered that graphene binding of single stranded nucleic acids exhibits dependency based on factors such as salt concentration, the type of salts present in the mixture, pH, temperature and the like. It has been found that the presence of multivalent salts of the form X²⁺, wherein X is generally the alkali earth elements such as Be, Mg, Ca, and Sr, promote more effective site binding of single stranded nucleic acids to graphene than monovalent salts of the form Y+, in which Y is generally one or more of the alkali metals such as, Li, Na, and K, but may also be buffers such as tris and ganidinium thiocyanate.

At concentrations of >0.2 mmol, single stranded nucleic acids have been found to exhibit graphene binding the presence of these X²⁺ cations at rates and efficiencies that are only achieved in the presence of Y⁺ cations at concentrations greater than 1M. Thus, single stranded nucleic acids can be made to bind and release from the surface of graphene by controlling the concentration of the X²⁺ cations. In some situations, it may be preferable to use Y⁺ cations at a higher concentration to cause single stranded nucleic acids to bind the graphene. One example of a Y⁺ cation is guanidinium thiocyanate. Without being bound to any theory, it is believed that Y⁺ cations such as guanidinium thiocyanate promote binding and helps lyse cells.

One non-limiting method for controlling the concentration of X²⁺ in the method as disclosed herein is to add a suitable chelating agent to the composition that preferentially binds X²⁺ cations. Non-limiting examples of such chelating agents include EDTA, BAPTA-AM, and/or EGTA which preferentially bind the X²⁺ cations. When release of bound single-stranded nucleic acid is desired, X²⁺ cations can be replaced with Y⁺ cations which affects the release of the single stranded nucleic acids from the surface of the magnetic beads as disclosed herein.

Another non-limiting method for controlling the concentration of X²⁺ or Y⁺ cations in the method as disclosed herein is to separate the solid bio-complex composed of the nucleic acids bound to the surface from the liquid with dissolved X²⁺ or Y⁺ cations and add water without X²⁺ or Y⁺ cations. This causes the single stranded nucleic acids to release into the water as the concentration of X²⁺ or Y⁺ cations falls below the critical threshold for binding.

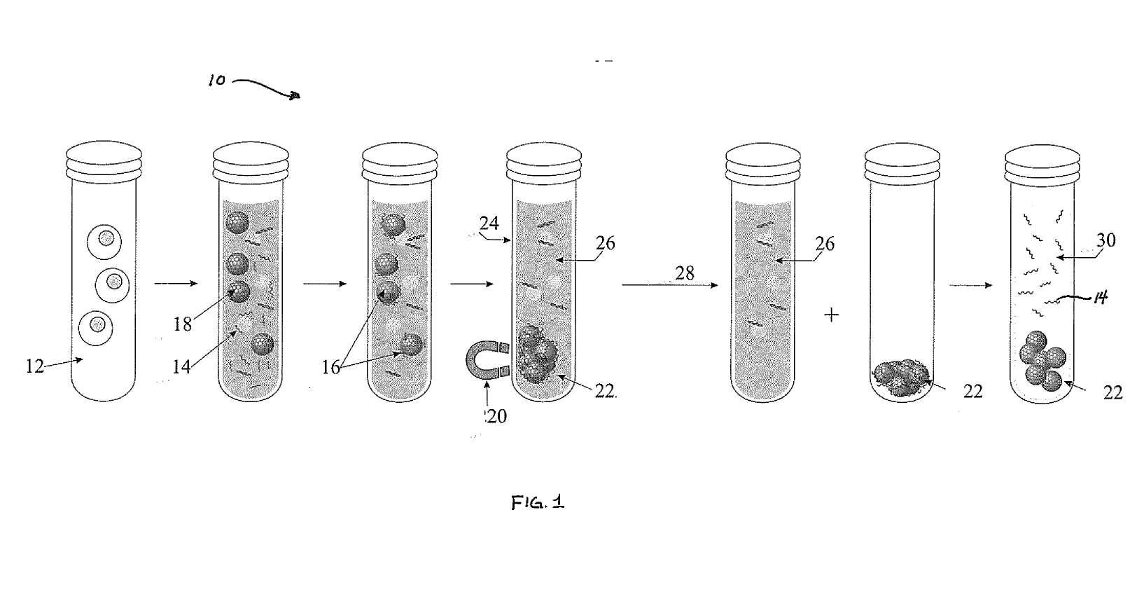

The general process disclosed is illustrated in FIG. 1 using mitochondrial RNA (mRNA) isolation as an example. In the method 10, a sample 12 believed to contain mRNA 14 is mixed or otherwise brought into contact with a suitable carbon coated material such as carbon coated magnetic beads as well as a suitable cationic compound; i.e. graphene magnetic nanoparticles (GMNP) and an X²⁺ material such as Ca²⁺ and/or a Y⁺ material such as guanidinium thiocyanate, respectively. The materials are permitted to interact for a time sufficient to promote bonding. In certain embodiments, the materials can be incubated at a suitable incubation temperature (i.e. between about 40° C. and about 70° C.) in the presence of the carbon-material coated magnetic beads 18 as disclosed herein for an interval sufficient to promote binding between the magnetic beads 18 and the miRNA 14 to form a bio-complex as at reference numeral 16.

The solution containing the resulting bio-complex can be subjected to a suitable magnetic field upon transfer to a suitable magnetic stand, depicted as a horseshoe magner as at reference numeral 20. The magnetic field is one that is sufficient to cause magnetic beads suspended in solution to compress to a smaller volume to form pellets 22 typically accumulated along the side 24 of the associated vial from the associated solution 26. The resulting material can be subjected to suitable separation techniques as at reference numeral 28 such as washing and elution to remove the pellets 22 from contact with the solution 26. The removed and isolated pellets 22 and/or the resulting solution 26 now considered a supernatant solution can be subjected to subsequent processing as desired or required. In certain applications, washing and elution of the pellets 22 can result in a solution 30 containing the miRNA strands 14.

The process as disclosed can be used to isolate and quantify the concentration of nucleic acids having a particular desired sequence. The graphene coated beads as disclosed bind only single stranded nucleic acids. Where it is desirable to determine if a single stranded nucleic acid with a particular sequence is present in a sample, nucleic acid with the complimentary sequence can be mixed into the sample, called the probe. In the situations where the target analyte is present, a duplex will form. In situations where the target analyte is absent, the excess single-stranded DNA probes will also bind to the beads and can be removed from solution. In this way, if a sample contains only single stranded nucleic acids, complimentary probes can be used to sequentially quantify specific sequences.

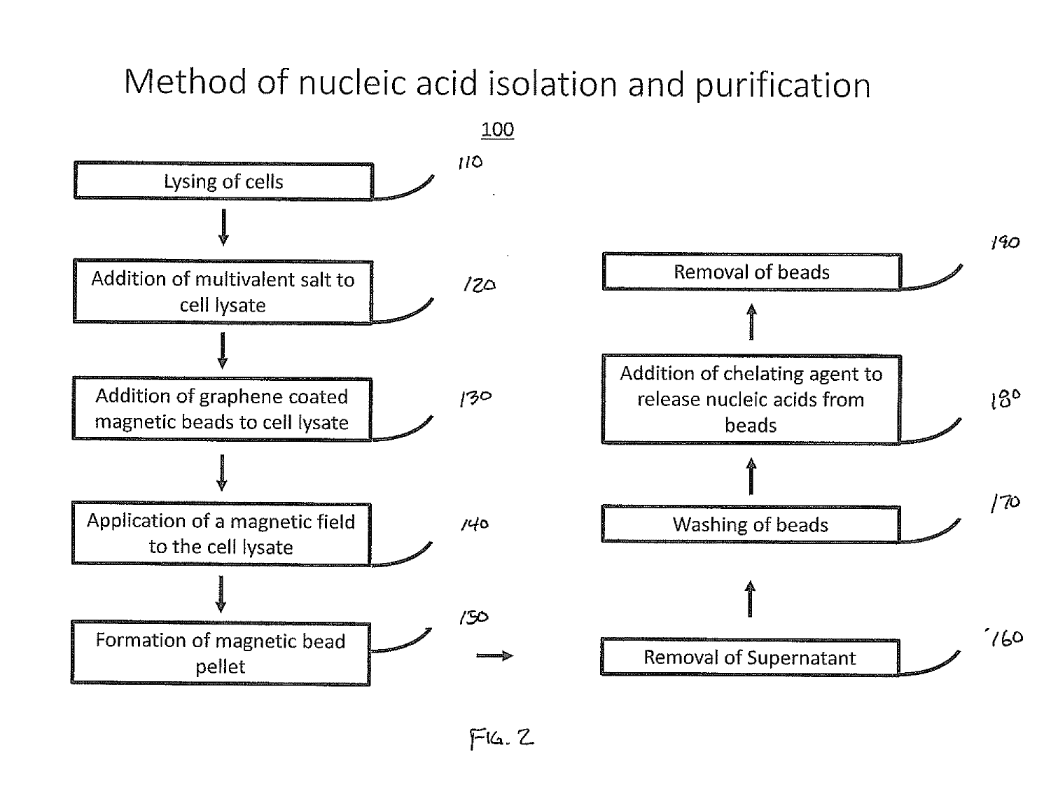

A flow chart of the process for isolating and/or purifying nucleic acids as disclosed herein is found in FIG. 2. A non-limiting example of the process for obtaining a sample of single stranded nucleic acids is depicted at reference numeral 100. A sample of cells having the nucleic acid material of interest can be collected, isolated and lysed by suitable techniques as at reference numeral 110 to produce a suitable cell lysate solution. The sample under analysis may be cells, blood, saliva, or any other biological substance and may also be any synthetic substance which contains nucleic acids. If the nucleic acids in the sample are contained in cells or other biological or synthetic structures, it may be necessary to liberate the nucleic acids by a chemical or physical method. For example, if the sample contains cells and the nucleic acids are located in the cells, then reagents such as SDS, protease K, or guanidinium thiocyanate may be used to break open the cells to release the nucleic acids as at reference numeral 110.

The process may proceed when the sample contains nucleic acids liberated from the confines of the cells or other envelopes referred to as the cell lysate solution. suspended in water. The group 2 or transition metal cations, salt, or guanidinium thiocyanate and mixtures thereof, called the binding buffer, and graphene magnetic beads may be added to the sample, at step 110 and 120, respectively. The multivalent cations as disclosed herein can be introduced to the cell lysate solution by suitable admixture of multivalent salt compounds as at reference numeral 120. The concentration of multivalent cations present in the cell lysate solution can be between 0.1 mM and 10M cationic compound. In the preferred implementation, the mixture contains 5 to 40 mmol of cationic compound such as X⁺ cations. Otherwise, the concentration of Y+ cations from compounds such as guanidinium thiocyanate should be present at concentrations greater than 1M and will be lower than 7M.

A non-limiting example of a multivalent cation salts which may be used at step 120 includes calcium chloride. The salt may be added as a solid or dissolved in a liquid. More generally, the multivalent cation added in step 120 may be any material which when mixed into water yields dissolved cations of the form X²⁺. Some examples of X include Ca, Mg, Be, and Sr. Further examples of salts which may be mixed into the solution to yield X²⁺ cations include calcium chloride, calcium citrate, calcium oxalate, calcium hydroxide, calcium phosphate, calcium sulfate. Any of these materials may be mixed into water to form a solution containing the X²⁺ cation which may be added in step 120. Alternatively, if the concentration of guanidinium thiocyanate used in step 110 is greater than 1M then the guanidinium thiocyanate may be sufficient to promote binding at step 120.

Once the multivalent cations are present in the cell lysate solution, carbon compound coated magnetic beads as disclosed herein can be added to the cell lysate solution as at reference numeral 130. In certain embodiments, the carbon coating with be composed in whole or in part of graphene. The carbon compound coated magnetic beads are generally suspended in the solution and are permitted to incubate for a time sufficient to form bio-complexes with single stranded nucleic acid material derived from the lysed cells.

One example of graphene coated magnetic beads is shown in FIG. 5. As depicted, the graphene coated magnetic beads are 1 nm to 100 microns in size and have a carbon layer 1 angstrom to 50 nm thick. The carbon layers can be visualized in FIG. 5 as rings around the magnetic metal core. The magnetic metal core may be any material where Fe, Co, or Ni individually or combined compose more than 30% of material by atomic percent.

In the embodiment depicted in FIG. 2, graphene magnetic beads are added to the sample at step 130. It is also within the purview of this disclosure that the process steps outlined at reference numerals 110, 120, and 130 may be done simultaneously or in another order. For example, if the sample is cells with nucleic acids contained in intact cells, a lysate such as a solution of 6M guanidinium thiocyanate combined with calcium chloride may be added to both lyse the cells and add the X²⁺ cation for step 120. Similarly, the sample may be added to the suspension of graphene magnetic beads. Before proceeding to step 140, the mixture may contain the nucleic acids from step 110, the X²⁺ cation from step 120, and the graphene magnetic beads from step 130. In this mixture, the nucleic acids bind to the magnetic beads to create the bio-complex.

Upon completing of a suitable incubation or binding interval, the cell lysate can be subjected to a suitable magnetic field as at reference numeral 140. The magnetic field applied is of sufficient intensity and duration to trigger the organization of the bio-complexed magnetic beads into magnetic bead pellets as at reference numeral 150. At step 140, the magnetic field can be applied to the mixture by placing a permanent magnet in proximity to the vial or container holding the lysate composition or by utilizing an electromagnet to generate a magnetic field within the volume of the vial. After the magnetic field is applied, the magnetic beads with the nucleic acids bound are pulled to the magnet forming a pellet or pellets in which the particles are sedimented near the magnet.

Formation of the magnetic bead pellets can be followed by removal of the resulting supernatant fluid as depicted at reference numeral 160. Removal can be accomplished by suitable methods. The supernatant can be retained for further analysis or discarded depending on the analysis underway.

In the process outlined in FIG. 2, the isolated bio-complexed magnetic beads can be washed or otherwise processed as at reference numeral 170 using suitable analytical techniques. It is contemplated that step 170 may be optional. In certain applications, the beads may be washed one or more times by addition of another liquid. This liquid may optionally also contain the X²⁺ cation and may contain other components such as a buffer like Trisaminomethane.

Once suitably prepared, the bio-complexed magnetic beads can be introduced into a suitable solution to which at least one chelating agent can be added to affect release of the complexed nucleic acids from association with the carbon coated magnetic beads as at reference numeral 180. At Step 180, a solution containing a chelating agent is added to sequester the X²⁺ cation. An example of this chelating agents is ethylenediaminetetraacetic acid, more commonly known as EDTA. Other chelating agents such as ethylene glycol tetraacetic acid (EGTA) may also be used. More generally, any aminopolycarboxilic acid, meaning any compound containing one or more nitrogen atom connected though carbon atoms to two or more carboxyl groups. Known calcium chelators also include 1,2-Bis(2-aminophenoxy) ethane-N,N,N′,N′-tetraacetic acid tetrakis(acetoxymethyl ester), also known as BAPTA-AM. Further examples of calcium chelating agents could also include any molecule containing one or more carboxylic acid groups. Alternatively, pure water may be added to cause release of the complexed nucleic acids. The nucleic acids will be released if the concentration of X²⁺ cations such as Ca²⁺ or Y⁺ cations such as the guanidinium cation are below the necessary concentration to cause binding.

The carbon coated magnetic beads can be removed from the solution by any suitable method as at reference numeral 190 leaving a solution containing the isolated nucleic acid material that can be analyzed and/or processed as desire or required. In certain applications, it is believed that the isolated carbon-coated magnetic beads may be suitable for recycle and reuse in subsequent operations. At step 190, the beads may be removed by applying a magnetic field again as in step 140 and removing the supernatant containing the purified nucleic acids.

It is contemplated that the process and material as disclosed herein can be employed is to store RNA long term. RNA is known to be fragile and to degrade upon exposure to various enzymes and proteins extant in the environment. One non-limiting example of such material is ribonuclease (RNase). It has been found that single-strand nucleic acids such as RNA bind to the surface of the carbon-coated magnetic beads as disclosed herein in a manner that renders them resistant to interaction with enzymes such as RNase. Thus the bio-complex of a single-strand nucleic acid such as RNA and the carbon coated magnetic beads as disclosed herein can provide a stable long-term storage vector for fragile single-strand nucleic acids.

It is contemplated that the stable stored single-strand nucleic acid material can comprise 10-5000 ng of nucleic acids per microliter and 50-500,000 ng of carbon coated material per microliter, wherein the carbon coated material is preferably magnetic or silica nanoparticulate for this embodiment. The suspension for storage contains between 0.1 mM and 10M cationic compound and is an aqueous suspension. The single-strand nucleic acid, while on the surface of the carbon-based material such as graphene, cannot be cleaved by enzymes such as RNase and will not spontaneously degrade.

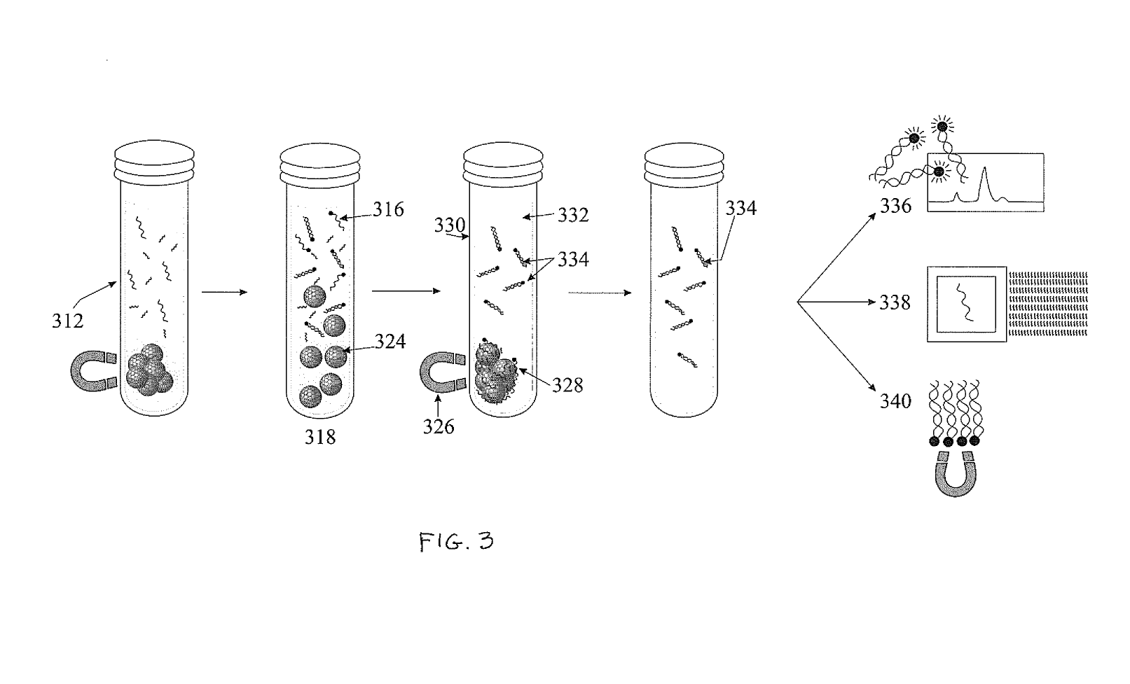

The method as disclosed herein can also be employed to isolate and quantify a single stranded nucleic acid material. A non-limiting exemplary method is illustrated in FIG. 3. A biological material to be analyzed is collected as at reference numeral 312. The sample in question can contain target single stranded nucleic acids such as miRNA 314 as well as other single stranded nucleic acid material. Specific DNA probes 316 can be added to the sample as illustrated at reference numeral 318 and allowed to react with any complementary nucleic acid material present in the sample 314 to form a duplex containing both the probe and the target which does not bind the carbon coated material in the presence of the cationic compound. After a suitable reaction interval, graphene magnetic nanoparticles (GMNP), or another suitable carbon coated material, 320 as disclosed herein can be added to the sample along with the cationic compound as described in step 120. The GMNP 320 can selectively bind to nucleic acids present in the sample which are not a duplex as at reference numeral 322. The resulting complex 324 can be subjected to a suitable magnetic field as at reference numeral 326 resulting in the formation of pellets 328 forming on one or more side surfaces of the associated vial 330 leaving a supernatant liquid 332 containing duplex nucleic acid material 334 which incorporates the target and the complementary nucleic acid material. Separation of the supernatant liquid from contact with the pellets 328 yields a solution of the duplex material 334 that is amenable to various analytic techniques including, but not limited to florescent tag detection 336, simple PCR 338, magnetic sensor detection 340 and the like.

Aspects of the disclosed implementations also include a method for isolating a nucleic acid having a specific sequence. The method is illustrated in process 400. At step 410, a sample containing single stranded nucleic acids may be obtained in order to assay a single-stranded nucleic acid of interest, for example a total RNA assay. In certain applications, it is contemplated that the sample may be one derived from the process outlined in FIG. 2. At step 420, a complimentary nucleic acid probe can be added to the sample. The DNA probe that is added to the sample will typically be one that is complementary to the RNA of interest, i.e. complimentary base pairs to the RNA base pairs A, T, G, and C would be the DNA base pairs T, A, C, and G, respectively.

At Step 430, a material composed of one or more multivalent salts is added to the sample. The multivalent salt(s) can be those outlined previously in this disclosure and can be added in the manner as described in step 120. Graphene coated magnetic beads are added to the sample as at Step 440. In the method as disclosed, Steps 430 and 440 may be combined or interchanged without affecting the outcome of the process.

Once the graphene coated magnetic beads and multivalent salts are added, a magnetic field is applied at Step 450. The magnetic field can be applied in the same manner as described previously in step 140. If the target of interest is present in the sample, the resulting supernatant liquid may contain double stranded nucleic acids produced as a result of the addition of the nucleic acid probe. The double stranded nucleic acids in the supernatant may then be amplified by PCR. Also, detection of the double stranded nucleic acids may be quantified with or without PCR by any known method for quantifying nucleic acids.

If it is desirable to quantify more than one nucleic acid, then the pellet that results from the application of the magnetic field as describe previously and remains after removal of the supernatant liquid in step 460 may be further processed as in Step 480. In step 480, the solutions from step 180 can be used to release the nucleic acids from the surface of the magnetic beads into the associated liquid. At step 490, the process returns to step 420 and repeats as many times as are required. On repeat steps, step 440 may be omitted as it is contemplated that the magnetic beads already resident in the solution can be re-utilized.

Aspects of the disclosed implementations also include a method for fabricating the carbon coated magnetic beads that can be employed in the method and process disclosed herein. One such fabrication method is discussed in US Published Application Number 2015-0170807 to Hagedorn, K. and Malizia, H, the specification of which is incorporated by reference herein. The referenced patent discloses methods for preparing magnetic beads. The magnetic beads prepared in carbon containing organic solvents such as xylenes or toluene may create a carbon coating on the surface of the magnetic beads suitable for use in the disclosed methods.

While the invention has been described in connection with what is presently considered to be the most practical and preferred embodiment, it is to be understood that the invention is not to be limited to the disclosed embodiments but, on the contrary, is intended to cover various modifications and equivalent arrangements included within the spirit and scope of the appended claims, which scope is to be accorded the broadest interpretation so as to encompass all such modifications and equivalent structures as is permitted under the law.

What is claimed is:

1. A method for separating single stranded nucleic acids from associated biological material, the method comprising:
creating a complex of a carbon coated material and single stranded nucleic acids from a mixture containing nucleic acids, carbon coated material and multivalent cations, wherein the cations employed include at least one of multivalent salts at concentrations from 5 mmol to 7M, monovalent cations at concentrations from 1M to 7M;
after the complex has been created, removing the carbon coated material complexed with single-stranded nucleic acid from the mixture.

2. The method of claim 1 wherein the carbon coating is at least one of graphene, pyrolytic carbon or a mixture of graphene and pyrolytic carbon.

3. The method of claim 1 wherein the multivalent cations are alkali earth metals or alkali earth metal salts, wherein the multivalent cations are present in an aqueous solution.

4. The method of claim 1 wherein the monovalent cations are guanidinium ions.

5. The method of claim 1 wherein the multivalent cations are Ca²⁺ ions in an aqueous solution present in an aqueous solution.

6. The method of claim 1 wherein the carbon coated material is configured as beads, the beads each having a core and an outer surface wherein at least the core is magnetic and contains at least 30% by weight of a metal selected from the group consisting of Ni, Fe, Co, or mixtures thereof.

7. The method of claim 6 wherein the removing step includes subjecting the resulting complex of carbon-coated magnetic beads and nucleic acid to at least one of a magnetic field, a centrifugal force, precipitation, or mixtures thereof.

8. The method of claim 1 wherein the carbon coated material comprises a substrate and an outer carbon coating, the carbon coating having a thickness between 1 angstrom and 50 nm.

9. The method of claim 1 further comprising the step of:
after removing the carbon coated magnetic beads from the complex, releasing single-stranded nucleic acid from attachment to the carbon coated material, wherein the releasing step occurs with addition of a chelating agent into contact with the removed carbon coated material.

10. The method of claim 9 wherein the chelating agent is an aminopolycarboxylic acid selected from the group consisting of EDTA, BAPTA-AM, EGTA and mixtures thereof and wherein the chelating agent is present as a solid or as an aqueous solution to provide a concentration of 1 mmol to 100 mmol.

11. The method of claim 1 further comprising the step of:
after removing the carbon coated magnetic beads from the complex, releasing single-stranded nucleic acid from attachment to the carbon coated material, wherein the releasing step occurs with addition of water into contact with the removed carbon coated material.

12. The method of claim 1 wherein the carbon-coated material is configured as individual beads, each bead having an interior core and an outer surface, the beads composed of one of the following: a magnetic metal containing at 30% by weight of a metal selected from the group consisting of Ni, Fe, Co, or mixtures thereof, silica or polymeric substrates, and wherein the carbon coating is present as a carbon layer on at least a portion of the outer surface of the bead, the carbon layer comprising at least one of graphene, pyrolytic carbon or a mixture of graphene and pyrolytic carbon, and wherein the multivalent cations are alkali earth metals or alkali earth metal salts or mixtures thereof.

13. A method for isolating a single stranded nucleic acid with a specific sequence comprising:
admixing a composition containing a mixture of single stranded nucleic acids with complimentary nucleic acid probes for an interval sufficient for the complimentary nucleic acid probes to make a duplex with complimentary sequences in the mixture of single stranded nucleic acids; and
adding to the resulting admixture, carbon-coated material, complimentary nucleic acid probes and monovalent or multivalent cations for an interval sufficient to permit association between the carbon coated material and at last a portion the single stranded nucleic acid, wherein the association may include all nucleic acids from the original mixture which did not form a duplex with the nucleic acid probes and the nucleic acid probes which did not form a duplex; and
removing the carbon coated magnetic material and associated single stranded nucleic acid from contact with the mixture.

14. The method of claim 13 wherein the removal step includes at least one of the following: centrifugation, precipitation, subjecting the composition to a magnetic field.

15. The method of claim 13 wherein the complementary nucleic acid probe is a chain of nucleic acids selected such that the chain of nucleic acids contains complimentary bases with corresponding positions to the single stranded nucleic acid that is to be isolated or purified.

16. The method of claim 13 wherein the complementary nucleic acid probe may be DNA or a synthetic nucleic acid.

17. The method of claim 16 further comprises the step of quantifying the sequence for study by performing PCR on the supernatant which remains after removal of carbon coated magnetic material and associated single stranded nucleic acids.

18. The method of claim 13 wherein the complimentary strand further comprises a fluorescent marker.
